(19) United States
(12) Patent Application Publication
(10) Pub. No.: US 2021/0275128 A1
(43) Pub. Date: Sep. 9, 2021

(54) Breakaway Stethoscope

(71) Applicant: James Salazar, Riverside, CA (US)

(72) Inventor: James Salazar, Riverside, CA (US)

(21) Appl. No.: 17/194,744

(22) Filed: Mar. 8, 2021

Related U.S. Application Data
(60) Provisional application No. 62/986,323, filed on Mar. 6, 2020.

(51) Int. Cl. A61B 7/02

(52) U.S. Cl. CPC A61B 7/02; A61B 2560/0443

(57) ABSTRACT

A stethoscope designed with a breakaway feature to prevent strangulation when worn by medical professionals is described. The stethoscope's sound-channeling tube contains at least one weakness which makes it less likely for the tube to remain in a single piece when exposed to the amount of force required to cause harm.

### CROSS REFERENCE TO RELATED APPLICATIONS

The present application claims the benefit of provisional application Ser. No. 62/986,323, filed Mar. 6, 2020, by the present inventor.

### FIELD OF INVENTION The present invention relates to stethoscopes and more particularly to abuse resistant stethoscope designs.

### BACKGROUND

Any article worn around one's neck may be a choking hazard. In particular, a stethoscope may present significant danger to medical professionals. Recent years have seen a sharp increase in violence against medical professionals. Several such cases have involved medical professionals being strangled, or harmed in another way, by their own stethoscopes. Patients in emergency situations may be disoriented and perceive the medical professional as an attacker. Patients with mental health diseases may turn violent.

What is needed is a simple, effective and affordable stethoscope that can be safely worn by medical professionals while reducing the risk of the stethoscope being used to endanger the professional.

### SUMMARY OF INVENTION

The present invention introduces a novel stethoscope with inexpensive breaking points manufactured into the structure of the tubing itself. These breaking points may take the form of sections of tubing with particularly thin, or otherwise delicate, walls. Attempts to use the stethoscope for strangulation should fail as the tubing would separate before it could cause harm to any individual.

### DESCRIPTION OF THE DRAWINGS

FIG. 1 shows a drawing of a stethoscope with a single weak point.

FIG. 2 shows a section of tubing with integrated weak points.

FIG. 3 shows a section of tubing with partial perforations.

FIG. 4 shows a section of tubing that may be separated and reconnected.

FIG. 5 shows a length of tubing with weak points of varying strength.

### DETAILED DESCRIPTION

The majority of stethoscopes used by medical professionals are of two types: Littmann or Sprague-Rappoport. Such stethoscopes comprise a chest piece, a headset, and tubing which connects the two. In Littmann-style stethoscopes, one tube connects the chest piece to the headset, and sound is transmitted to two. The headset comprises two earpieces, which are connected to the tube by a binaural. In Sprague-Rappoport-style stethoscopes, two tubes transmit sound from the chest piece, with one tube connected to each earpiece. Sprague-Rappoport stethoscopes often comprise a junction at which the two tubes are joined, such that the tubes are neater and are less likely to catch on surrounding objects. A tube may also be referred to as a lumen. Stethoscope designs other than Littmann or Sprague-Rappoport may be used in accordance with the present invention.

It is a goal of the invention that major modification to conventional stethoscope design would not be necessary, both to simplify manufacturing and aid adoption by practitioners. Accordingly, the headset and chestpiece may be constructed as in any conventional stethoscope. The tubing of the present invention may be of the same material, thickness, and diameter of conventional stethoscopes. Alternatively, these parameters may be altered such that the tubing itself fails at a particular force. However, it is preferred that the weak point be constructed in the tubing as a structure rather than a material. In particular, the tube may be constructed from a vinyl, such as polyvinyl chloride, a latex rubber, or a soft plastic.

Stethoscopes constructed in accordance with some embodiments of the present invention may be intended for a single use only. Single-use, or single-patient, stethoscopes are commonly used by first-responders and by practitioners in situations where patients are in isolation. Single-use stethoscopes may be constructed in Littmann or Sprague-Rappoport styles. Single-use stethoscopes mainly differ from conventional stethoscopes in the materials used, in order to lower costs. The cost effectiveness of the designs described herein may come, in part, from the reduced number of components required to achieve the safety benefit, when compared to prior efforts.

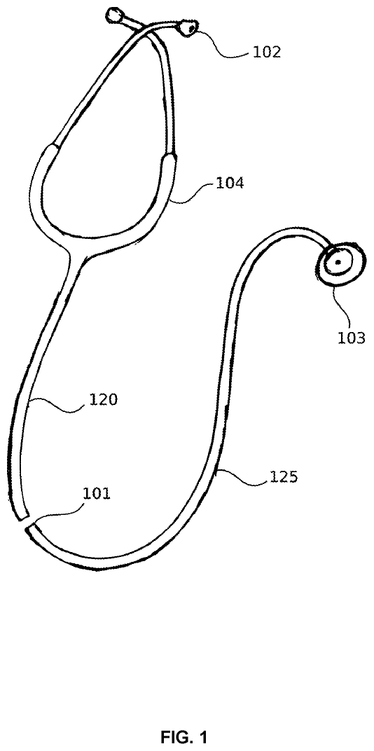

FIG. 1 shows a stethoscope consistent with the present invention. A chest piece 103 receives an audio signal and pases it via tubing 120, 125 to a headset 104 and ultimately to earpiece 102. In the embodiment presented in FIG. 1, a single weak point 101 (shown separated) is built into the tubing, causing the tubing to separate if a threshold level of force is applied to it, thus preventing the tubing from being used to cause harm.

The weak point 101 may be constructed at any point in the tubing or at multiple points. It is preferred that the weak point be constructed at roughly the midpoint of the tubing, minimizing the resulting length of tubing after the tubing breaks. Minimizing the resulting length may increase the safety of the device.

The weak point can be constructed in many ways. Any construction that results in mechanical failure of the tube at a predictable force is sufficient. Potential constructions are perforations 203 within the tubing and variable wall thickness. Variable wall thickness may be considered an indentation in the tube The tubing may also be constructed in two pieces and bonded together using another material, such that the bond has the desired failure characteristics.

The failure force may be in the range 4-14 pounds of force, preferably between 8-10 pounds. Danger begins at 4 PSI, at which point jugular veins may be blocked. Assuming an average neck size 15 inches in circumference and a tube that is ½ inch diameter, a force of approximately 30 pounds would be damaging. In the worst-case scenario, a neck size of 10 inches and tube of ¼ inch diameter corresponds to 10 pounds of force causing harm. The exerted pressure increases roughly linearly as tubing diameter decreases, and as such, very narrow tubing is not preferred. The selected failure force will typically be a trade-off between safety and durability. A very low failure force may prevent any harm from being inflicted using the stethoscope tubing, but the stethoscope tubing may fail under ordinary use, or if the tubing gets caught or pulled on a surrounding object.

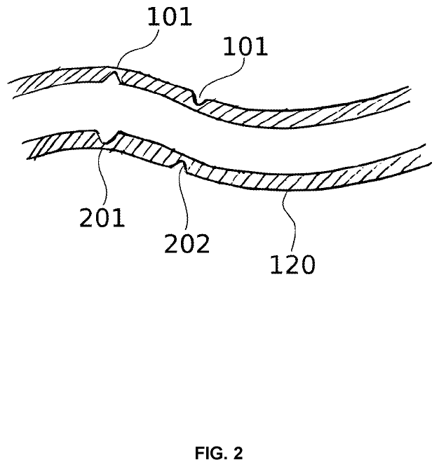

FIG. 2 shows a section of tube with two integrated weak points 101. In this example, one weak point is formed onto the outside surface 202 of the tube and one weak point is formed onto the inside surface of the tube 201. A goal of the invention is to preserve sound quality compared to traditional stethoscopes. To this end, the weak point 202 may be constructed such that the tube tapers on the outer edge, leaving the inner tube unchanged. However, it may be desired that the weak point be discreet and invisible from the outside as with internal tapers/indentations 201. The reduction in sound quality may be imperceivable, and be worth the discreteness.

The present invention may be embodied in any type of acoustic (non-electronic) stethoscope, including Littman and Sprague-Rappoport style stethoscopes. The invention may be adapted to suit stethoscopes with two lumens or two tubes, such as Sprague-Rappoport style stethoscopes. In this case, weak points may be constructed in both tubes and the failure force for each tube may be adapted such that the two-tube system preserves the desired fail force. Preferably, the weak point should be located below the junction of the tubes in the case of Sprague-Rappoport stethoscopes. In the case of Littmann stethoscopes, the weak point should preferably be located below the headset.

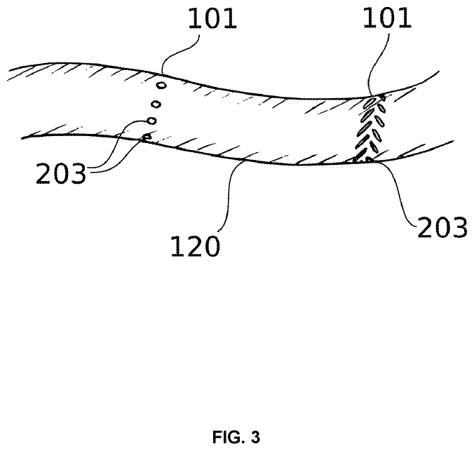

FIG. 3 shows a section of tube with two weak points 101. In this example, the weak points take the form of indentations or partial perforations 203. These partial perforations may be formed in patterns to enable the tube to be separated with specific amounts of force. In some embodiments, the tube may be made of multiple layers of material. Outer layers of the tube may be completely perforated, while the inner layers may not be perforated. In this way, desired structural properties may be obtained without interfering with the desired audio properties.

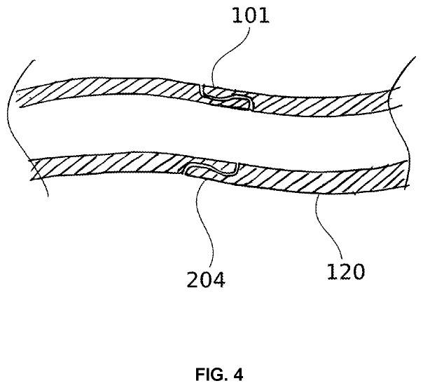

FIG. 4 shows an example of a section of tube incorporating a reattachable weak point 204. In this example, the tube has a structure formed within it that releases upon the application of a sufficient force. In this case, however, the tube is not permanently damaged and the sections may be reassembled for continued use. The weak connection 204 used may be a tube-to-tube mechanical connection as shown in FIG. 4, a press-fit friction connection, an adhesive connection or any other type of connection that does not require additional structures.

Generally, it is preferred that stethoscope tubing is kept as short as possible to preserve sound quality. Stethoscopes commonly are available in 22-inch and 27-inch varieties, in which the length is measured from the top of the headset to the bottom of the chest piece. With these standard stethoscopes lengths, one weak point in the tubing will likely be sufficient. However, the tubing may be constructed with multiple weak points 101 along the tube as in FIG. 2, such that the tubing may fail at additional points should the tubing be used as a weapon after a first failure. In the case of stethoscopes with tubing longer than standard, two or more weak points may be used. In a preferred embodiment, the maximum length after failure is 10 inches—short enough to prevent an attacker from encircling a neck and having enough tube length remaining to leverage and cause harm.

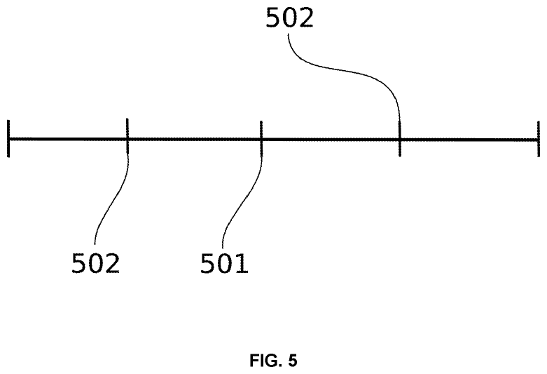

FIG. 5 Shows a length of tube that is subdivided by multiple weak points 501, 502. Here, the weakest point 501 is located approximately at the midpoint of the length of tube. This point is expected to fail first, providing the largest reduction in overall tube length. Additional weak points 502 are then located at approximately the one quarter and three quarter points on the length of tube. These points are designed to fail, but not as readily as the primary fail point 501 because the length of tube bisected by these points is shorter and considered to be less of a risk. This multiple point of failure scheme also enables a medical professional to balance the risk of the tube being used to harm against the risk of the tube separating during ordinary use. The medical professional may, for example, apply tape to the circumference of the weakest point 501 to make it less likely to separate in ordinary use, knowing that the secondary fail points 502 may still provide some safety from the risk of intentional harm.

The above examples and discussion are provided so that one skilled in the art would understand how to create and benefit from the invention. The examples provided are not intended to be limiting. Therefore, the spirit and scope of the appended claims should not be limited to the description of the preferred versions contained herein.

I claim:

1. A stethoscope comprising:
an earpiece;
a chest-piece;
a sound channeling tube operable to move sound from said chest-piece to said earpiece; and
at least one weak point formed within said sound channeling tube;
whereby said at least one weak point is operable to separate said sound channeling tube into at least two shorter tube portions if said sound channeling tube is pulled tightly.

2. The stethoscope of claim 1, wherein said weak point is an indentation.

3. The stethoscope of claim 2, wherein said indentation is internal.

4. The stethoscope of claim 2, wherein said indentation is external.

5. The stethoscope of claim 1, wherein said weak point is a perforation.

6. The stethoscope of claim 1, wherein said weak point has a failure force of at least four pounds.

7. The stethoscope of claim 1, wherein said weak point is reconnectable to said sound channeling tube.

8. The stethoscope of claim 1, wherein said sound channeling tube comprises an inner layer of material and an outer layer of material.

9. The stethoscope of claim 8, wherein said outer layer of material provides specifically limited separation resistance and said inner layer of material provides desired acoustic properties.

10. The stethoscope of claim 1, wherein a substantial length of said sound channeling tube is uniformly weak.

11. The stethoscope of claim 1, wherein the length of said sound channeling tube is subdivided by progressively stronger weak points such that the weakest point in the length is in the approximate midpoint along the length of said sound channeling tube and a slightly stronger weak point is located approximately at both the one quarter length and three quarter length points of said sound channeling tube.
